(19) United States
(12) Patent Application Publication
(10) Pub. No.: US 2022/0401170 A1
(43) Pub. Date: Dec. 22, 2022

(54) CONTROL UNIT, MEDICAL SYSTEM, AND CONTROL METHOD

(71) Applicant: TERUMO KABUSHIKI KAISHA, Tokyo (JP)

(72) Inventors: Yusuke SEKINE, Chigasaki-shi (JP); Yuki SAKAGUCHI, Isehara-shi (JP)

(73) Assignee: TERUMO KABUSHIKI KAISHA, Tokyo (JP)

(21) Appl. No.: 17/822,495

(22) Filed: Aug. 26, 2022

Related U.S. Application Data
(63) Continuation of application No. PCT/JP2021/002982, filed on Jan. 28, 2021.

(30) Foreign Application Priority Data
Feb. 27, 2020 (JP) 2020-032304

(51) Int. Cl. A61B 34/37; H04N 7/18

(52) U.S. Cl. CPC A61B 34/37; H04N 7/181; A61B 2034/303

(57) ABSTRACT

A control unit including a master control apparatus that is able to transmit a master signal for providing overall control of a plurality of operating devices capable of operating medical instruments used at a medical site, and a hub control apparatus that receives the master signal from the master control apparatus, and provides motion control of the operating devices according to the master signal. The hub control apparatus provides coordinated control of the plurality of operating devices according to the master signal.

### CROSS-REFERENCES TO RELATED APPLICATIONS

This application is a continuation of International Application No. PCT/JP2021/002982 filed on Jan. 28, 2021, which claims priority to Japanese Application No. 2020-032304 filed on Feb. 27, 2020, the entire content of both of which is incorporated herein by reference.

### TECHNOLOGICAL FIELD

The present disclosure generally relates to a control unit, a medical system, and a control method.

### BACKGROUND DISCUSSION

Catheter interventions have long been performed for various kinds of medical treatment with medical instruments, such as a guide wire, a balloon catheter, and a stent, inserted to an in-vivo location in a patient's body. In recent years, catheter robots for performing such medical practice have been progressively developed. For example, disclosed in Japanese Patent Laid-open No. 2017-205546 and Japanese National Publication of International Patent Application No. 2017-526397 are catheter robots that enable a surgeon, such as a doctor, to operate a medical instrument at a distance from a patient during a surgery in order to reduce the amount of radiation exposure of the surgeon.

The catheter robot described in Japanese Patent Laid-open No. 2017-205546 has a cassette in which a medical instrument is set. The cassette includes an arm, a first axis drive mechanism, a second axis drive mechanism, and a rotary drive mechanism. The arm has multiple degrees of freedom for properly positioning the medical instrument with respect to a patient. The first axis drive mechanism moves a guide wire back and forth along a longitudinal axis. The second axis drive mechanism moves various catheters back and forth along the longitudinal axis. The rotary drive mechanism rotationally drives the medical instrument around the longitudinal axis.

Meanwhile, the catheter robot described in Japanese National Publication of International Patent Application No. 2017-526397 has a plurality of delivery modes to enable a guide section for delivering a medical instrument to operate in a low or high speed mode for the purpose of smoothly moving the guide section in an in-vivo lumen of a patient.

The catheter robots described in Japanese Patent Laid-open No. 2017-205546 and Japanese National Publication of International Patent Application No. 2017-526397 are installed at a medical site (e.g., in a surgery room). A surgeon controls a catheter robot on a one-to-one basis by operating, for example, a controller from a workstation. Therefore, in a case, for example, where a surgery is performed by using a plurality of catheter robots differing in product specifications, such as the catheter robots described in Japanese Patent Laid-open No. 2017-205546 and Japanese National Publication of International Patent Application No. 2017-526397, the surgery cannot be performed continuously by using the workstation to operate the catheter robots simultaneously or alternately. Further, it is difficult to perform the surgery continuously by using the catheter robots together with another operating device designed for a different use.

Particularly, when produced by different manufacturers, the catheter robots are usually designed to use different user interfaces. Therefore, the surgeon needs to acquire proficiency in the use of such different user interfaces designed by different manufacturers, which can place an increased burden on the surgeon.

### SUMMARY

The present disclosure has been made to provide a control unit, a medical system, and a control method that enable a workstation to freely select and control a plurality of operating devices for operating medical instruments.

According to an aspect of the present disclosure, a control unit is disclosed that includes a master control apparatus configured to transmit a master signal for providing overall control of a plurality of operating devices configured to operate medical instruments at a medical site, and a hub control apparatus configured to receive the master signal from the master control apparatus and to provide motion control of the operating devices according to the master signal. The hub control apparatus is configured to provide coordinated control of the plurality of operating devices according to the master signal.

According to another aspect of the present disclosure, a medical system is disclosed that includes a plurality of operating devices configured to operate medical instruments at a medical site, a master control apparatus configured to transmit a master signal for providing overall control of the plurality of operating devices, and a hub control apparatus configured to receive the master signal from the master control apparatus and to provide motion control of the plurality of operating devices according to the master signal. The hub control apparatus provides coordinated control of the plurality of operating devices according to the master signal.

According to still another aspect of the present disclosure, a control method is disclosed, which includes transmitting a master signal from a master control apparatus to a hub control apparatus, and causing the hub control apparatus to provide coordinated control of a plurality of operating devices according to the master signal, the operating devices being able to operate medical instruments at a medical site.

The present disclosure is configured such that a hub control apparatus provides coordinated control of each of a plurality of operating devices according to a master signal transmitted from a master control apparatus. Therefore, a user is able to freely select and control the operating devices by using a workstation.

### BRIEF DESCRIPTION OF THE DRAWINGS

FIG. 1 is a diagram schematically illustrating a medical system according to an embodiment of the present disclosure.

FIG. 2 is a block diagram briefly illustrating a hub control apparatus according to the embodiment of the present disclosure.

FIG. 3 is a flowchart illustrating a control method according to the embodiment of the present disclosure.

### DETAILED DESCRIPTION

Set forth below with reference to the accompanying drawings is a detailed description of embodiments of a control unit, a medical system, and a control method. Note that since embodiments described below are preferred specific examples of the present disclosure, although various technically preferable limitations are given, the scope of the present disclosure is not limited to the embodiments unless otherwise specified in the following descriptions.

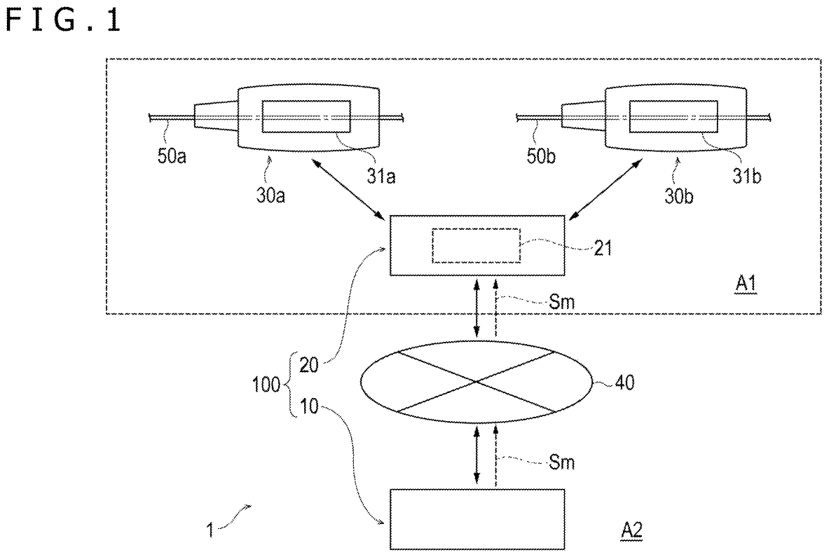

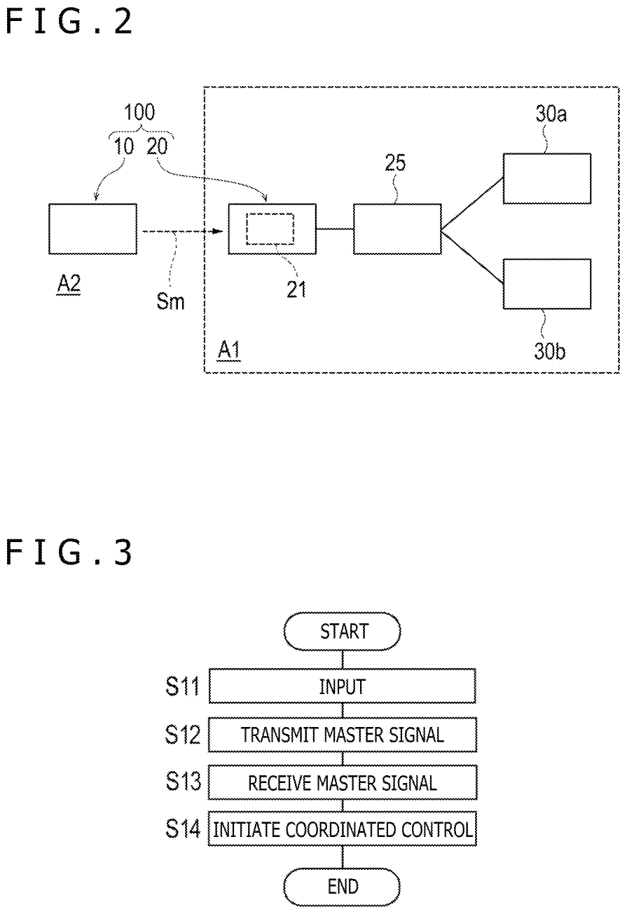

FIG. 1 is a schematic diagram illustrating a medical system 1 according to an embodiment of the present disclosure. FIG. 2 is a block diagram illustrating the medical system 1.

The medical system 1 is outlined below with reference to FIG. 1. The medical system 1 can include a first remote operating device (corresponding to an “operating device”) 30a, a second remote operating device (corresponding to an “operating device”) 30b, a master control apparatus 10, and a hub control apparatus 20. The first remote operating device 30a is able to operate a medical instrument 50a used at a medical site. The second remote operating device 30b is able to operate a medical instrument 50b used at the medical site. The master control apparatus 10 is able to transmit a master signal Sm that provides overall control of a plurality of remote operating devices 30a and 30b. The hub control apparatus 20 receives the master signal Sm from the master control apparatus 10, and provides motion control of the remote operating devices 30a and 30b according to the master signal Sm. The hub control apparatus 20 provides coordinated control of each of the plurality of remote operating devices 30a and 30b according to the master signal Sm.

The medical system 1 will now be described in detail.

In the present embodiment, the master signal Sm transmitted from the master control apparatus 10 is received by the hub control apparatus 20 in a treatment room A1. The remote operating devices 30a and 30b are remotely operated in the treatment room A1 by the hub control apparatus 20.

The master control apparatus 10 and the hub control apparatus 20 are configured to form a control unit 100 that provides coordinated control of each of the remote operating devices 30a and 30b.

The hub control apparatus 20 can mediate communication connection between the remote operating devices 30a and 30b and the master control apparatus 10. The hub control apparatus 20 can be installed, for example, in a space A1 such as a treatment room (surgery room) where medical practice is performed on patients. However, the specific location of the space A1 is not particularly limited to any kind.

Each of the remote operating devices 30a and 30b can be used, for example, in the treatment room A1, as is the case with the hub control apparatus 20.

The master control apparatus 10 may be installed in a space A2 that is different from the treatment room A1 where the medical instruments 50a and 50b are used for treatment purposes. The different space A2 can be, for example, a control room located near or remote from the treatment room A1. However, the specific location of the space A2 is not particularly limited to any kind.

The master control apparatus 10 and the hub control apparatus 20 are able to communicate with each other through a network 40 that includes a wired or wireless communication interface. The network 40 is not limited to any particular means as long as the network 40 is means capable of providing secure communication.

The hub control apparatus 20 can be mechanically connected to and disconnected from the remote operating devices 30a and 30b through a connection mechanism 25 (see FIG. 2) included in the hub control apparatus 20. According to the master signal Sm transmitted from the master control apparatus 10, the connection mechanism 25 is able to selectively connect and disconnect the hub control apparatus 20 to and from the first remote operating device 30a and selectively connect and disconnect the hub control apparatus 20 to and from the second remote operating device 30b.

For example, in a case where the first remote operating device 30a and the second remote operating device 30b are to be simultaneously operated during a predetermined surgery, the connection mechanism 25 connects the hub control apparatus 20 to each of the remote operating devices 30a and 30b. Meanwhile, in a case where only one of the first and second remote operating devices 30a and 30b is to be operated during a predetermined surgery, the connection mechanism 25 is able to mechanically disconnect the hub control apparatus 20 from a remote operating device that is not to be operated.

The connection mechanism 25 included in the hub control apparatus 20 can include, for example, an actuator that operates according to the master signal Sm.

The hub control apparatus 20 can include, for example, a drive section 21 that supplies a driving force to driving sources 31a and 31b included in the remote operating devices 30a and 30b, respectively. The drive section 21 is not particularly limited to any kind, and can include, for example, a well-known servomotor. The drive section 21 and the connection mechanism 25 may be accommodated in a predetermined housing, as is the case with a well-known switching hub.

The master control apparatus 10 can include user interfaces such as a video display monitor, a joystick, a microphone, and a motion sensor. In response to an input operation performed on the user interfaces by an operator such as a surgeon or an assistant to the surgeon, the master control apparatus 10 transmits the master signal Sm for moving back and forth or rotating the medical instruments 50a and 50b set on the remote operating devices 30a and 30b.

The master control apparatus 10 is further able to receive information regarding an ongoing surgery, for example, from the remote operating devices 30a and 30b and other medical devices used for surgical manipulation. The master control apparatus 10 can include an output section that causes, in real time, the received information to be displayed on a monitor and reproduced as a sound output, for example, from a speaker.

The first remote operating device 30a and the second remote operating device 30b each include a robot that delivers the medical instruments 50a and 50b to a desired in-vivo location in a patient's body according to a control signal transmitted from the master control apparatus 10 to the hub control apparatus 20 through the network 40.

Each of the remote operating devices 30a and 30b may include, for example, a built-in camera capable of capturing still images and video images. The remote operating devices 30a and 30b are each able to transmit information, such as video images captured by a camera, to the master control apparatus 10 through the network 40. By transmitting the information such as video images, the remote operating devices 30a and 30b are able to feed back the state of an actual operation of each of the remote operating devices 30a and 30b and the state of a patient during a surgery to a surgeon and an assistant to the surgeon.

The operation target of the first remote operating device 30a can include the medical instrument 50a which can be relatively long (elongated) and insertable to an in-vivo location in a patient's body. The medical instrument 50a is not particularly limited to any kind, and can be, for example, a guide catheter, a guide wire, a balloon catheter, a stent delivery system, an atherectomy catheter, or other catheter devices.

The operation target of the second remote operating device 30b can include the medical instrument 50b which is relatively long (elongated) and insertable to an in-vivo location in a patient's body, as is the case with the first remote operating device 30a. The medical instrument 50b is not particularly limited to any kind, and can be, for example, a guide catheter, a guide wire, a balloon catheter, a stent delivery system, an atherectomy catheter, or other catheter devices.

The first remote operating device 30a can include the driving source 31a which operationally drives the medical instrument 50a. The driving source 31a can include, for example, transport means such as an operating roller for operationally driving the medical instrument 50a to move the medical instrument 50a back and forth or rotate the medical instrument 50a.

The second remote operating device 30b can include the driving source 31b which operationally drives the medical instrument 50b. As is the case with the driving source 31a, the driving source 31b can include, for example, transport means such as an operating roller for operationally driving the medical instrument 50b to move the medical instrument 50b back and forth or rotate the medical instrument 50b.

In the medical system 1 according to the present embodiment, the first remote operating device 30a and the second remote operating device 30b may operate on different devices, that is, operate on the medical instruments 50a and 50b that differ from each other. More specifically, in a case where the medical instrument 50a selected as the operation target of the first remote operating device 30a is one of the guide catheter, the guide wire, the balloon catheter, the stent delivery system, and the atherectomy catheter, the medical instrument 50b selected as the operation target of the second remote operating device 30b from among the guide catheter, the guide wire, the balloon catheter, the stent delivery system, and the atherectomy catheter may be different from the medical instrument 50a selected as the operation target of the first remote operating device 30a.

Further, in the medical system 1 according to the present embodiment, the first remote operating device 30a and the second remote operating device 30b may differ in product specifications. More specifically, the first remote operating device 30a and the second remote operating device 30b may differ, for example, in manufacturer, component structures, and operation.

As described above, even in a case where a plurality of remote operating devices to be controlled include a plurality of types of remote operating devices 30a and 30b differing in the medical instruments 50a and 50b to be operated and/or a plurality of types of remote operating devices 30a and 30b differing in product specifications, the hub control apparatus 20 provides coordinated control according to the master signal Sm transmitted from the master control apparatus 10, which helps enable the surgeon to perform smooth surgical manipulation continuously by using the remote operating devices 30a and 30b.

The term “coordinated control” in this document denotes that selectively driving or stopping the remote operating devices 30a and 30b at any timing requested by the surgeon or the assistant during a predetermined surgical manipulation. An example of coordinated control is providing motion control for driving the second remote operating device 30b to deliver, for example, the balloon catheter to a treatment area in a blood vessel along the guide wire while driving the first remote operating device 30a to move or properly position the guide wire.

During a surgical manipulation performed by using the remote operating devices 30a and 30b, the master control apparatus 10 is able to transmit the master signal Sm that contains a command for reporting the operating status of each of the remote operating devices 30a and 30b to the surgeon and the assistant. The remote operating devices 30a and 30b each can include a reporting section that reports, to the location where the remoting operating devices 30a, 30b are located(e.g., operating room), the operating status according to the master signal Sm received from the master control apparatus 10. The reporting section can include devices (e.g., a speaker, a display, and a lamp) that are capable of outputting, for example, a sound, a video image, and light. Upon receiving the command for reporting the operating status to the outside from the master control apparatus 10 through the hub control apparatus 20, the remote operating devices 30a and 30b operate the reporting section according to the operating status. For example, the reporting section included in a currently operating one of the remote operating devices 30a and 30b performs a reporting operation (e.g., illuminates a lamp) for its surroundings, which makes it relatively easy for the surgeon and the assistant to identify, on time, a device connected to the master control apparatus 10 during a surgery.

A control method executed by the medical system 1 will now be described. FIG. 3 is a flowchart illustrating the control method.

In S11, the surgeon or the assistant inputs, to the master control apparatus 10, a motion command based on the surgery to be performed.

In S12, the master control apparatus 10 transmits the master signal Sm to the hub control apparatus 20.

In S13, the hub control apparatus 20 receives the master signal Sm.

In S14, the hub control apparatus 20 begins to provide coordinated control of the remote operating devices 30a and 30b according to the master signal Sm.

In the medical system 1 according to the present embodiment, the plurality of remote operating devices 30a and 30b on which the medical instruments 50a and 50b are set can be parallelly motion-controlled or selectively motion-controlled according to the master signal Sm transmitted from the master control apparatus 10, which helps save the surgeon and the assistant from having to acquire proficiency in the use of the user interfaces of the plurality of control apparatuses individually linked to the plurality of remote operating devices 30a and 30b.

Further, for example, the surgeon and the assistant can be able to selectively use different types of medical instruments 50a and 50b at an appropriate timing during a surgical manipulation by using the plurality of remote operating devices 30a and 30b, which helps enable the surgeon to continuously perform a relatively smooth surgical manipulation.

Moreover, for example, the use of the plurality of remote operating devices 30a and 30b differing in product specifications helps enable the surgeon to continuously perform a surgery while selectively using the remote operating devices 30a and 30b. For example, in a case where the first remote operating device 30a manufactured by Company X is suitable for moving the guide wire to a lesion at a relatively high speed while the second remote operating device 30b is suitable for moving the guide wire to a lesion accurately at a relatively low speed (i.e., as speed lower the relatively high speed), it is possible to roughly move the guide wire to a location near a patient's lesion by using the first remote operating device 30a, and then accurately move the guide wire to a location near the patient's lesion by using the second remote operating device 30b. By adopting the above-described control method, the surgeon is able to continue performing a surgical manipulation more smoothly and improve treatment outcomes.

In addition, the medical system 1 allows the hub control apparatus 20 to discretionarily and selectively transmit and receive information to and from the remote operating devices 30a and 30b. Therefore, the medical system 1 also makes it possible to share information regarding a surgery and centralize organized information regarding a patient's condition. Additionally, in case of trouble or an emergency, such as the failure of a remote operating device 30a or 30b, the medical system 1 is able to use a normal remote operating device as a substitute for the faulty remote operating device, which helps ensure that the medical system 1 is redundant and safe.

Furthermore, the master control apparatus 10 can be used in combination with a desired number of remote operating devices. Therefore, the medical system 1 can be excellent in extensibility (i.e., being designed to allow the addition of new capabilities or functionality), which helps lower the level of proficiency that is required of the surgeon engaged in a surgery according to the product specifications for the remote operating devices and the product specifications for the control apparatuses.

The control unit, the medical system, and the control method according to the preferred embodiment of the present disclosure have been described in this document. However, the present disclosure is not limited to the preferred embodiment described in this document. Various modifications can be made by persons skilled in the art without departing from the scope of the appended claims.

In the foregoing description, devices for operating relatively long (elongated) medical instruments such as catheters are cited as the examples of the operating devices. The reason is described below. Catheter treatment is currently performed in such a manner that the surgeon discretionarily combines various types of devices for treatment purposes according to a patient's condition (a bloodstream, the shape and properties of a lesion, etc.) that varies from one patient to another. Therefore, specifications for currently available catheter robots (operating robots), which accept only predetermined combinations of applicable medical instruments, are merely applicable to some cases of illness. In view of such a problem, the embodiment has been described with reference to an example application where the catheters are used for treatment purposes. However, specific types of applicable operating devices are not particularly limited to any type as long as they are used to operate appropriate medical instruments.

Further, the operating devices are not limited to devices that are remotely operable by an operator such as the surgeon or the assistant. For example, the operating devices may be configured to be directly operable by the operator in a space such as a treatment room.

Moreover, the medical instruments to be operated by the operating devices are not limited to relatively long (elongated) medical instruments such as catheters. A variety of well-known medical instruments are applicable in the medical field. For example, even in hybrid surgery in which minimally invasive treatment and conventional surgical treatment are combined, various operating devices are devised to improve treatment outcomes and patient quality of life (QOL). Under these circumstances, it is possible that an operating device (catheter operating robot) devised for percutaneous coronary intervention (PCI) is used together with a surgery robot for hybrid surgery. In such a case, newly developing a robot for hybrid surgery can be too costly and is therefore irrational. The master control apparatus and the hub control apparatus according to the embodiment of the present disclosure are able to provide coordinated control without regard to the type of operating device, which helps eliminate the necessity of newly developing and introducing a control apparatus for hybrid surgery, and can reduce development cost and use cost.

In addition, the control unit 100 may include two or more master control apparatuses 10. In a case where the control unit 100 includes two or more master control apparatuses 10, coordinated control can be provided by using a discretionary combination of two or more master control apparatuses and two or more operating devices. For example, in a case where a first operating device includes two or more driving sources to be controlled and a second operating device includes two or more driving sources to be controlled, one master control apparatus 10 is able to transmit, to the hub control apparatus 20, the master signal for controlling two or more freely-selected driving sources of the first and second operating devices, whereas another master control apparatus 10 is able to transmit, to the hub control apparatus 20, the master signal for controlling other freely-selected driving sources of the first and second operating devices. When two or more master control apparatuses 10 transmit the master signal to provide coordinated control of two or more driving sources of two or more operating devices as described above, a plurality of surgeons and assistants are able to collaboratively perform surgery. In the above exemplary embodiment, for example, a plurality of surgeons differing in the experience in performing a predetermined surgical manipulation are able to control the driving and stopping of operating devices at an appropriate timing based on the situation of an ongoing surgery. Therefore, surgery can be performed more smoothly and safely than in a case where the operating devices are coordinately controlled by only one surgeon or only one assistant.

Further, it is sufficient if the number of operating devices to be controlled is two or more, and the number of operating devices to be controlled is not limited to any particular number.

Furthermore, the space where the operating devices are used are not limited, for example, to a treatment room. Moreover, for enhanced security of and reduced communication delay in the medical system, an alternative configuration may be adopted by configuring the operating devices for edge computing, configuring the master control apparatuses for cloud computing, or using a data diode as an appropriate data transmission system between the control apparatuses and operating devices in the medical system.

The detailed description above describes embodiments of a control unit, a medical system, and a control method. These disclosed embodiments represent examples of the control unit, the medical system, and the control method disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A control unit comprising:
a master control apparatus configured to transmit a master signal for providing overall control of a plurality of operating devices configured to operate medical instruments at a medical site; and
a hub control apparatus configured to receive the master signal from the master control apparatus and to provide motion control of the operating devices according to the master signal, and wherein the hub control apparatus is configured to provide coordinated control of the plurality of operating devices according to the master signal.

2. The control unit according to claim 1, wherein the plurality of operating devices include a device configured to be remotely operated by an operator and/or a device configured to be directly operated by the operator.

3. The control unit according to claim 1, wherein an operation target of the plurality of operating devices includes a medical instrument configured to be inserted into a living body.

4. The control unit according to claim 3, wherein the plurality of operating devices include a driving source configured to operationally drive the medical instrument.

5. The control unit according to claim 4, wherein the hub control apparatus is configured to coordinately control motion of the driving source for the plurality of operating devices according to the master signal.

6. The control unit according to claim 1, wherein the plurality of operating devices include a plurality of types of operating devices differing in the medical instruments to be operated and/or a plurality of types of operating devices differing in product specifications.

7. The control unit according to claim 1, wherein the master control apparatus is installed in a space that is different from a treatment room where the medical instruments are used for treatment purposes.

8. The control unit according to claim 7, wherein the hub control apparatus is configured to receive, in the treatment room, the master signal transmitted from the master control apparatus.

9. The control unit according to claim 8, wherein the plurality of operating devices are configured to be operated in the treatment room by the hub control apparatus.

10. The control unit according to claim 1, wherein one or more of the plurality of operating devices is a robot configured to control a catheter during a percutaneous coronary intervention.

11. The control unit according to claim 1, wherein one or more of the plurality of operating devices include a camera configured to capture images that convey one or more of a state of an operational procedure and a state of a patient.

12. The control unit according to claim 1, wherein the medical instruments include one or more of a guiding catheter, a guide wire, a balloon catheter, a stent delivery system, and an atherectomy catheter.

13. The control unit according to claim 1, wherein the coordinated control of the plurality of the operating devices includes selectively driving or stopping the plurality of operating devices during a surgical procedure.

14. The control unit according to claim 13, wherein coordinated control of the plurality of the operating devices is configured to provide motion control to deliver a balloon catheter to a treatment area in a blood vessel and to move or position a guide wire in the blood vessel.

15. The control unit according to claim 1, wherein the master signal includes a command for reporting an operating status of the plurality of operating devices to an external device.

16. A medical system comprising:
a plurality of operating devices configured to operate medical instruments at a medical site;
a master control apparatus configured to transmit a master signal for providing overall control of the plurality of operating devices; and
a hub control apparatus configured to receive the master signal from the master control apparatus and to provide motion control of the plurality of operating devices according to the master signal, and wherein the hub control apparatus is configured to provide coordinated control of the plurality of operating devices according to the master signal.

17. A control method comprising:
transmitting a master signal from a master control apparatus to a hub control apparatus; and
causing the hub control apparatus to provide coordinated control of a plurality of operating devices according to the master signal, the operating devices being configured to operate medical instruments at a medical site.

18. The control method according to claim 17, further comprising:
remotely operating the plurality of operating devices by an operator or directly operating the plurality of operating devices by the operator.

19. The control method according to claim 17,
wherein an operation target of the plurality of operating devices includes a medical instrument configured to be inserted into a living body;
the plurality of operating devices include a driving source configured to operationally drive the medical instrument; and
wherein the hub control apparatus is configured to coordinately control motion of the driving source for the plurality of operating devices according to the master signal.

20. The control method according to claim 17, wherein the plurality of operating devices include a plurality of types of operating devices differing in the medical instruments to be operated and/or a plurality of types of operating devices differing in product specifications.
